(19) United States
(12) Patent Application Publication
(10) Pub. No.: US 2021/0247354 A1
(43) Pub. Date: Aug. 12, 2021

(54) AMMONIA DETECTOR

(71) Applicant: DENSO CORPORATION, Kariya-city (JP)

(72) Inventors: Eriko MAEDA, Kariya-city (JP); Takumi OKAMOTO, Kariya-city (JP); Hiroaki YOTO, Kariya-city (JP); Toshihiko HARADA, Nisshin-city (JP)

(73) Assignee: DENSO CORPORATION, Kariya-city (JP)

(21) Appl. No.: 17/245,784

(22) Filed: Apr. 30, 2021

Related U.S. Application Data
(63) Continuation of application No. PCT/JP2019/040554, filed on Oct. 16, 2019.

(30) Foreign Application Priority Data
Oct. 30, 2018 (JP) 2018-203601

(51) Int. Cl. G01N 27/407

(52) U.S. Cl. CPC G01N 27/4071; G01N 27/4075

(57) ABSTRACT

An ammonia detector includes a solid electrolyte body, a detection electrode, and a reference electrode. The solid electrolyte body includes a measured gas facing surface, which comes into contact with a measured gas, and a reference gas facing surface, which comes into contact with a reference gas. The detection electrode is formed on the measured gas facing surface of the solid electrolyte body. The reference electrode is formed on the reference gas facing surface of the solid electrolyte body. The detection electrode contains at least alumina and 50% or more by mass of Pd.

### CROSS-REFERENCE TO RELATED APPLICATION

The present application is a continuation application of International Application No. PCT/JP2019/040554 filed on Oct. 16, 2019, which is based on and claims the benefit of priority from Japanese Patent Application No. 2018-203601 filed Oct. 30, 2018. The contents of these applications are incorporated herein by reference in their entirety.

### BACKGROUND

The present disclosure relates to an ammonia detector that detects the concentration of ammonia.

Gas sensors have been used to monitor the combustion state of internal combustion engines and the operation of exhaust gas treatment devices. Such a gas sensor is located in, for example, an exhaust gas passage and detects the concentration of various gases contained in exhaust gas.

A urea selective catalytic reduction (SCR) system that reduces NOx (that is, nitrogen oxides) contained in the exhaust gas has been known as an exhaust gas treatment device. The urea-SCR system includes an aqueous urea solution injector, which is located upstream of a selective reduction NOx catalyst and supplies an aqueous urea solution that produces a reducing agent, which is ammonia. The amount of aqueous urea solution to be supplied is controlled based on, for example, the detection result of a NOx sensor, which is located downstream of the selective reduction NOx catalyst. To efficiently reduce and purify NOx, the aqueous urea solution needs to be supplied without deficiency or excess. For this reason, in addition to the concentration of NOx in the exhaust gas that has passed through the selective reduction NOx catalyst, the concentration of ammonia is desirably detected to be reflected in feedback control. An ammonia detector has therefore been developed that detects the concentration of ammonia in the exhaust gas.

### SUMMARY

One aspect of the present disclosure provides an ammonia detector including a solid electrolyte body, a detection electrode, and a reference electrode. The detection electrode contains at least alumina and 50% or more by mass of Pd.

Reference signs in parentheses in the scope of claims indicate the correspondence to specific means disclosed in the embodiments described below and do not limit the technical scope of the present disclosure.

### BRIEF DESCRIPTION OF THE DRAWINGS

The above-mentioned and other objects, features and advantages of this disclosure will become more apparent by reference to the following description taken in conjunction with the accompanying drawings, in which

FIG. 1 is a longitudinal sectional view of a detecting element showing the main section of an ammonia detector according to a first embodiment;

FIG. 2 is a cross-sectional view taken in the direction of arrows along line II-II of FIG. 1;

FIG. 3 is a schematic diagram illustrating three-phase interfaces on a cross-section of a detection electrode containing a solid electrolyte component according to the first embodiment;

FIG. 4 is a schematic diagram illustrating three-phase interfaces on a cross-section of a detection electrode containing alumina according to the first embodiment;

FIG. 5 is a schematic diagram illustrating an exhaust gas purifying system of an internal combustion engine to which the ammonia detector of the first embodiment is applied;

FIG. 6 is an explanatory diagram showing mixed potentials of the detection electrode according to the first embodiment;

FIG. 7 is a cross-sectional schematic diagram illustrating a device for evaluating the performance in detecting ammonia using a simplified element in Experimental Example 1;

FIG. 8 is a graph showing the relationship between the ammonia sensitivity of Pd, Rh, Au, Ir, and Pt and the ammonia concentration in Experimental Example 1;

FIG. 9 is a graph showing the relationship between the NO sensitivity of Pd, Au, and Pt and the NO concentration in Experimental Example 1;

FIG. 10 is a graph showing the relationship between the NO sensitivity of Rh, Au, and Ir and the NO concentration in Experimental Example 1;

FIG. 11 is a graph showing the relationship between the NO₂ sensitivity of Pd, Au, and Pt and the NO₂ concentration in Experimental Example 1;

FIG. 12 is a graph showing the relationship between the NO₂ sensitivity of Rh, Au, and Ir and the NO₂ concentration in Experimental Example 1;

FIG. 13 is an explanatory diagram illustrating a simulation model of Experimental Example 1;

FIG. 14 is an explanatory diagram illustrating adsorption sites in the simulation model of Experimental Example 1;

FIG. 15 is an explanatory diagram illustrating a calculation model of an O₂ dissociative adsorption energy Eo in Experimental Example 1;

FIG. 16 is an explanatory diagram illustrating the relationship between the O₂ dissociative adsorption energy and the NH₃ sensitivity of noble metals in Experimental Example 1;

FIG. 17A is an explanatory diagram illustrating a calculation model of a cohesive energy EA in a simulation model including only Au atoms in Experimental Example 1, and FIG. 17B is an explanatory diagram illustrating a calculation model of a cohesive energy E_{B} in a simulation model including only Pd atoms in Experimental Example 1;

FIG. 18 is an explanatory diagram showing the cohesive energy of Au and Pd in Experimental Example 1;

FIG. 19 is a schematic diagram illustrating an evaluation device for measuring oxidizing power of noble metals with respect to ammonia in Experimental Example 2;

FIG. 20 is an explanatory diagram showing the analysis result of Au obtained by a temperature-programmed reaction method in Experimental Example 2;

FIG. 21 is an explanatory diagram showing the analysis result of Pd obtained by the temperature-programmed reaction method in Experimental Example 2;

FIG. 22 is a graph showing the relationship between the ammonia sensitivity and the ammonia concentration regarding Pd and Au in Experimental Example 3;

FIG. 23 is a graph showing the relationship between the NO sensitivity and the concentration of NO regarding Pd and Au in Experimental Example 3;

FIG. 24 is a graph showing the relationship between the NO₂ sensitivity and the NO₂ concentration regarding Pd and Au in Experimental Example 3;

FIG. 25 is an explanatory diagram showing the sensitivity of the detection electrode containing alumina to NO, NO₂, or ammonia in Experimental Example 4;

FIG. 26 is an explanatory diagram showing the sensitivity of the detection electrode containing YSZ to NO, NO₂, or ammonia in Experimental Example 4; and

FIG. 27 is an explanatory diagram showing errors in the ammonia sensitivity of the detection electrode containing alumina and the detection electrode containing YSZ in Experimental Example 4.

### DETAILED DESCRIPTION OF THE PREFERRED EMBODIMENTS

The ammonia detector includes a detection electrode for detecting the concentration of ammonia. The detection electrode is, for example, an Au electrode. Unfortunately, since the Au electrode detects NOx in addition to ammonia, the selectivity of gas detection is low. Additionally, due to a low melting point, Au cannot be fired together with other ceramic materials during manufacturing processes. As a result, an additional process for forming the Au electrode such as plating needs to be performed, which increases the manufacturing costs. Furthermore, the fact that Au has a low melting point means that the heat resistance of the Au electrode is insufficient, and the use of the Au electrode as the detection electrode may possibly cause Au to evaporate from the detection electrode.

To solve these problems, for example, JP 2017-116371 A proposes a detection electrode that is formed of an alloy of Pt and Au with Pt serving as the main component. This increases the selectivity to ammonia gas and additionally raises the melting point of the detection electrode, which increases the heat resistance.

An ammonia detector including a conventional detection electrode is still susceptible to improvement in the detection accuracy of ammonia. For example, the alloy of Pt and Au used for the detection electrode undesirably oxidizes ammonia due to excessively high reactivity to ammonia. As a result, ammonia that should be detected by the detection electrode is consumed. That is, the ammonia detector with the conventional detection electrode is hindered from accurately detecting the concentration of ammonia in terms of the ammonia oxidation activity.

Furthermore, the alloy of Pt and Au also reacts with NOx though not as much as Au does. As a result, the detection electrode formed of the alloy of Pt and Au reflects the concentration of NOx in the measurement result besides the concentration of ammonia. That is, the ammonia detector with the conventional detection electrode is hindered from accurately detecting the concentration of ammonia also in terms of the selectivity to ammonia in the exhaust gas.

The present disclosure aims at providing an ammonia detector that includes a detection electrode with a high heat resistance and additionally is capable of accurately detecting the concentration of ammonia.

One aspect of the present disclosure provides an ammonia detector including a solid electrolyte body, a detection electrode, and a reference electrode. The solid electrolyte body includes a measured gas facing surface, which comes into contact with a measured gas, and a reference gas facing surface, which comes into contact with a reference gas. The detection electrode is formed on the measured gas facing surface of the solid electrolyte body. The reference electrode is formed on the reference gas facing surface of the solid electrolyte body. The detection electrode contains at least alumina and 50% or more by mass of Pd.

The detection electrode of the ammonia detector contains at least alumina (aluminum oxide) and 50% or more by mass of Pd. Such a detection electrode has a high melting point and maintains the electrode performance under a high-temperature environment of, for example, 1400° C. Thus, the ammonia detector including the detection electrode configured as described above can be formed by integrally firing the detection electrode with other members constituting the ammonia detector and formed of, for example, ceramic material during manufacturing. This enables the manufacturing of the ammonia detector at a low cost. Additionally, the evaporation of the electrode component from the detection electrode is inhibited, which increases the heat resistance of the detection electrode. That is, the ammonia detector is excellent in durability under high-temperature environments.

The detection electrode configured as described above inhibits consumption of ammonia by oxidation while keeping the reactivity to ammonia to a degree such that the concentration of ammonia is accurately detected. As a result, since the ammonia detector detects ammonia while reducing the consumption of ammonia by the detection electrode, ammonia is accurately detected.

The reason why the concentration of ammonia is accurately detected may also be as follows. While the reaction caused by the oxidation of ammonia and the reaction caused by the reduction of NO₂ tend to occur at the interface between Pd and the solid electrolyte body, reaction of NO is unlikely to occur. Since the detection electrode contains 50% or more by mass of Pd, the sensitivity to ammonia is increased while reducing the sensitivity to NO. Additionally, since the detection electrode contains alumina that has no oxygen ion conductivity, when NO₂ passes through the cavities in the detection electrode, NO₂ is easily decomposed to NO on the surface and the inside of the detection electrode. NO₂ is hindered from reaching the interface between the detection electrode and the solid electrolyte body, and NO generated by the decomposition of NO₂ reaches the interface. Thus, electromotive force or the like that results from the reaction of NO is unlikely to be produced between the detection electrode and the reference electrode, and electromotive force or the like that results from the reaction of ammonia becomes significant. Consequently, the ammonia detector accurately detects the concentration of ammonia even under an environment in which, for example, NOx (that is, nitrogen oxide) coexists.

As described above, the above aspect is excellent in the heat resistance of the detection electrode and additionally detects the concentration of ammonia accurately.

### First Embodiment

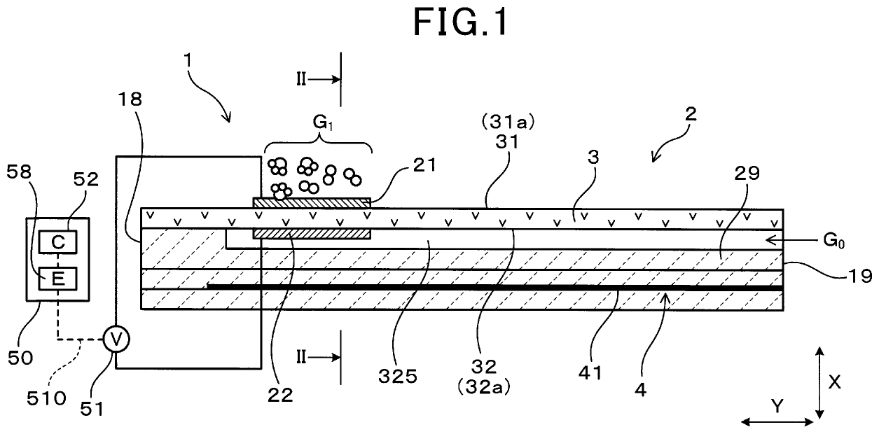

An ammonia detector according to an embodiment will be described with reference to FIGS. 1 to 8.

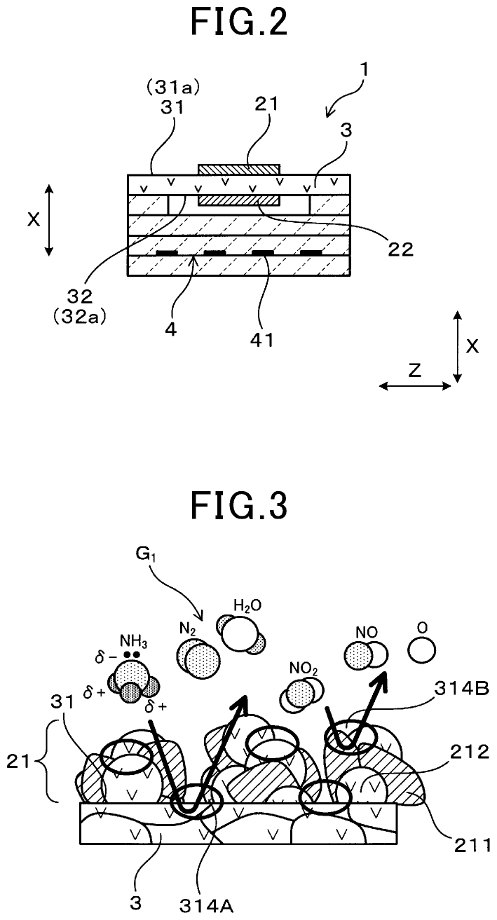

As illustrated in FIGS. 1 and 2, an ammonia detector 1 includes a detecting element 2, which is constituted by a laminate of, for example, a detection electrode 21, a reference electrode 22, a solid electrolyte body 3, and a heater 4. In the following description, the laminating direction of the detecting element 2 is referred to as an X direction, the longitudinal direction of the detecting element 2 orthogonal to the X direction is referred to as a Y direction, and the widthwise direction of the detecting element 2 orthogonal to the X direction and the Y direction is referred to as a Z direction.

The solid electrolyte body 3 includes a measured gas facing surface 31 and a reference gas facing surface 32. The measured gas facing surface 31 is a surface that comes into contact with a measured gas G₁. The reference gas facing surface 32 is a surface that comes into contact with a reference gas G₀. The measured gas G₁ is, for example, exhaust gas, and the reference gas G₀ is, for example, the atmospheric air. Hereinafter, the same reference sign is used for the measured gas and the exhaust gas and for the reference gas and the atmospheric air. The measured gas and the reference gas, however, are not limited to the exhaust gas and the atmospheric air, respectively. For example, both the measured gas G₁ and the reference gas G₀ may be exhaust gas. The measured gas facing surface 31 and the reference gas facing surface 32 may be different surfaces from each other as illustrated in FIGS. 1 and 2, but may also be, for example, surfaces that are continuous as if they are the same planar surface.

The solid electrolyte body 3 is, for example, an oxygen ion conductive solid electrolyte. Such a solid electrolyte includes, but not particularly limited to, for example, yttria-stabilized zirconia (hereinafter, referred to as YSZ).

The solid electrolyte body 3 is shaped like, for example, a plate or a rod but not particularly limited to these shapes. The solid electrolyte body 3, which is plate-shaped as illustrated in FIGS. 1 and 2, includes a first main surface 31a and a second main surface 32a on its surfaces. The first main surface 31a and the second main surface 32a are located on opposite sides of the solid electrolyte body 3.

In FIGS. 1 and 2, the first main surface 31a is the measured gas facing surface 31, and the second main surface 32a is the reference gas facing surface 32. Although the illustration of the structure is omitted, both the detection electrode 21 and the reference electrode 22 may be formed on a surface of the detecting element 2 such as the first main surface 31a.

As illustrated in FIGS. 1 and 2, the measured gas facing surface 31, on which the detection electrode 21 is formed, is preferably the first main surface 31a, and the reference gas facing surface 32, on which the reference electrode 22 is formed, is preferably the second main surface 32a, which is located on the opposite side to the first main surface 31a. In this case, the detection electrode 21 and the reference electrode 22 are easily exposed to different gas atmospheres. As a result, the reference potential is stabilized, which further increases the detection accuracy of the detection electrode 21 in detecting ammonia.

The measured gas facing surface 31 is the outer surface of the detecting element 2 and is exposed to the outside of the detecting element 2. The reference gas facing surface 32 is the inner surface of the detecting element 2 and faces a reference gas chamber 325. The reference gas chamber 325 is a region surrounded by the solid electrolyte body 3 and an insulating substrate 29.

The detection electrode 21 is located on the section of the measured gas facing surface 31 at a distal end 18 side in an X-axis direction. The distal end 18 is, for example, the end to be inserted in an exhaust gas pipe. The end opposite to the distal end 18 in the X-axis direction is a proximal end 19.

The detection electrode 21 contains a noble metal 211, which is substantially formed of Pd. This means that the noble metal 211 is allowed to contain metals or inevitable impurities that do not adversely affect the advantages of the present disclosure besides Pd. The content of other metals and inevitable impurities allowed in the noble metal 211 is, for example, 5% or less by mass. In terms of sufficiently achieving the advantages of the present disclosure, the content is preferably 1% or less by mass, and the noble metal 211 more preferably consists of Pd and inevitable impurities.

The main component of the noble metal 211 in the detection electrode 21 is Pd. The noble metal 211 may be formed of only Pd or may contain, for example, Au besides Pd. Since the main component of the noble metal 211 in the detection electrode 21 is Pd, the heat resistance of the detection electrode 21 is increased. Additionally, the accuracy in detecting ammonia is further improved in this case. This is probably because the consumption of ammonia in the detection electrode 21 is further reduced, and the sensitivity of the detection electrode 21 to NOx is further decreased.

The state in which the main component of the noble metal 211 is Pd refers to a state in which the content of Pd in the noble metal 211 is the greatest. In terms of further improving the heat resistance and the detection accuracy, the content of Pd in the noble metal 211 can be set to 50% by mass to 100% by mass. With the content of Pd set to 50% by mass or more and less than 100% by mass, the remaining component of the noble metal 211 can be, for example, Au, that is, the noble metal 211 of the detection electrode 21 can be an alloy of Pd and other noble metals.

The component of the detection electrode 21 is analyzed using, for example, an electron probe microanalyzer (hereinafter, referred to as EPMA) or X-ray photoelectron spectroscopy (hereinafter, referred to as XPS).

In terms of inhibiting densification of the electrode caused by cohesion of the noble metal after firing, the average particle size of the noble metal 211 is preferably 0.2 μm or more. In terms of further enhancing this advantage, the average particle size of the noble metal 211 is more preferably 0.5 μm or more, and further preferably 0.7 μm or more. In terms of increasing the specific surface area of the electrode and increasing gas reaction points to improve the sensitivity, the average particle size of the noble metal 211 is preferably 5 μm or less. In terms of further enhancing this advantage, the average particle size of the noble metal 211 is more preferably 2 μm or less, and further preferably 1.5 μm or less.

The average particle size of the noble metal 211 in the detection electrode 21 is measured by image analysis using a scanning electronic microscope (that is, SEM). More specifically, the average of the particle diameters (that is, the average particle size) of noble metal alloy particles on the SEM image of the detection electrode 21 is calculated by a linear intercept method using the image processing software “WinROOF” manufactured by MITANI Corporation. That is, the images of scanning electronic microscope (SEM) photos are acquired at a magnification of 5000 times, and straight lines (that is, measurement lines) are further drawn on the images to calculate the average length of the section of each straight line that crosses a noble metal particle. Note that the measurement lines that reach the edge of the image are not included. In calculating the average value, 100 measurement lines are drawn on each SEM image, and SEM images of different sections are analyzed so that the total number of the measurement lines will be 1000 or more. Note that, in a case in which the detection electrode 21 contains alumina, the measurement can be performed in the same manner.

The thickness of the detection electrode 21 is preferably 1 μm or more in terms of causing NO₂ to be easily decomposed to NO before reaching a three-phase interface 314A formed by the detection electrode 21 and the solid electrolyte body 3. In terms of enhancing this advantage, the thickness of the detection electrode 21 is more preferably 5 μm or more, and further preferably 7 μm or more. In terms of improving the detection accuracy in detecting ammonia by reducing the consumption of ammonia due to oxidation before reaching the three-phase interface 314A of the detection electrode 21, the thickness of the detection electrode 21 is preferably 50 μm or less. In terms of further reducing the consumption of ammonia due to oxidation, the thickness of the detection electrode 21 is more preferably 30 μm or less, and further preferably 20 μm or less.

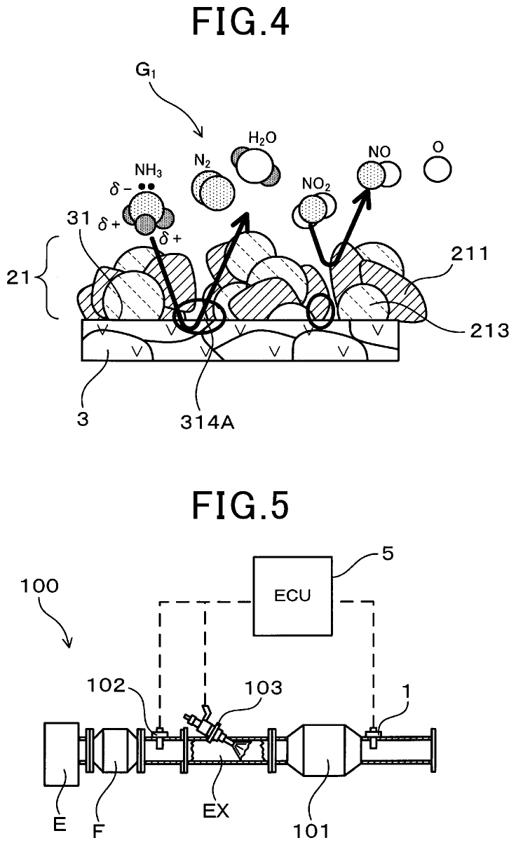

As illustrated in FIG. 3, the detection electrode 21 of the present embodiment contains the noble metal 211 and alumina 213. Additionally, the detection electrode 21 may contain a solid electrolyte component 212, which is identical to the material of the solid electrolyte body 3. The solid electrolyte component 212 may be similar to the component constituting the solid electrolyte body 3. The detection electrode 21 containing the solid electrolyte component 212 also forms a three-phase interface 314B by the solid electrolyte component 212, the noble metal 211, and a gas phase in the detection electrode 21. This increases the reactivity of the detection electrode 21 to ammonia. That is, in addition to the three-phase interface 314A, which is formed by the solid electrolyte body 3, the noble metal 211 in the detection electrode 21, and the gas phase, ammonia reacts at the three-phase interface 314B, which is formed by the solid electrolyte component 212 included in the detection electrode 21, the noble metal 211, and the gas phase. As a result, the ammonia sensitivity is improved. In FIGS. 3 and 4, the gas phase includes, for example, ammonia, nitrogen, water, nitrogen dioxide, nitric oxide, and oxygen and is represented by a space above the detection electrode 21 (more specifically, the upper section on the sheet of FIGS. 3 and 4). Additionally, cavities in the detection electrode 21 also correspond to the gas phase.

The noble metal 211 and the alumina 213 located on the surface of the detection electrode 21 facilitate dissociation of NO₂ in the gas phase to give NO. Since the main component of the noble metal 211 is Pd, in regard to the sensitivity of the detecting element 2 to NOx, the sensitivity to NO is lower than the sensitivity to NO₂. Thus, the selectivity of the detecting element 2 to ammonia is maintained.

If the detection electrode 21 does not contain the solid electrolyte component 212, ammonia reacts at the three-phase interface 314A formed by the solid electrolyte body 3, the detection electrode 21 (more specifically, the noble metal 211), and the gas phase as illustrated in FIG. 4. Some ammonia may possibly react before reaching the three-phase interface 314A. In the meantime, NOx such as NO₂ is likely to dissociate in the gas phase before reaching the three-phase interface 314A. As a result, the selectivity to ammonia is improved. The improvement of the selectivity is likely to be significant in a mixed-potential ammonia detector, which will be described below. If the detection electrode 21 contains alumina, the three-phase interface hardly exists on the surface of the detection electrode 21, and the dissociation of NO₂ is unlikely to contribute to the mixed potential. That is, in the ammonia detector 1 of a mixed-potential type, the detection electrode 21, which contains alumina, reduces the influence of the dissociation of NO₂ on the mixed potential. As a result, the selectivity to ammonia is further improved, which further improves the detection accuracy.

In terms of improving the selectivity to ammonia, the content of alumina 213 in the detection electrode 21 is preferably greater than that of the solid electrolyte component 212. In this regard, the content of the solid electrolyte component 212 in the detection electrode 21 is preferably less than the content of the alumina 213, more preferably 50% or less by mass, and further preferably, the detection electrode 21 substantially does not contain the solid electrolyte component 212. The state in which the detection electrode 21 substantially does not contain the solid electrolyte component 212 refers to a state in which the detection electrode 21 does not contain the solid electrolyte component 212 that is, for example, intentionally added. Regions such as those in which both components are mixed at the interface between the detection electrode 21 and the solid electrolyte body 3 are permitted.

The content of alumina in the detection electrode 21 can be 5 to 50% by mass. If the content of alumina is less than 5% by mass, NO₂ is unlikely to be decomposed to NO on the detection electrode 21. In contrast, if the content of alumina exceeds 50% by mass, the sensitivity of the detection electrode 21 to ammonia may possibly decrease.

A non-illustrated protection layer may be located on the surface of the detection electrode 21 to cover the detection electrode 21. In this case, the detection electrode 21 is protected from poisoning substances and flying matters in the exhaust gas G₁. The protection layer is formed of, for example, gas-permeable ceramic porous material. In this case, the porosity and the pore diameter of the ceramic porous material are desirably adjusted so that the exhaust gas G₁ promptly reaches the detection electrode 21.

The reference electrode 22 is formed on the reference gas facing surface 32 and is located on the opposite side of the solid electrolyte body 3 from the detection electrode 21. That is, the reference electrode 22 and the detection electrode 21 are located opposite to each other with the solid electrolyte body 3 located in between. The reference electrode 22 is formed in the reference gas chamber 325.

The reference electrode 22 is formed of a noble metal such as Pt. The reference electrode 22 may further contain a solid electrolyte component. The solid electrolyte component may be the same as the component constituting the solid electrolyte body 3.

A heater 4 is located close to the reference gas chamber 325 of the detecting element 2. The heater 4 is formed integrally with the detecting element 2 with a heating element 41 embedded in the insulating substrate 29, which defines the reference gas chamber 325.

The heating element 41 generates heat upon energization by electric power supply from a non-illustrated external power source to heat the detecting element 2 to a temperature appropriate for detecting ammonia. The insulating substrate 29 is formed of insulating ceramic such as alumina. The heating element 41 is laminated between unfired ceramic plates, on which the solid electrolyte body 3 is further laminated. The laminate is then fired to produce the detecting element 2 incorporating the heater 4.

As illustrated in FIG. 1, the temperature of the detecting element 2 is monitored by a current supply control section 58 and is controlled to be a predetermined operation temperature (hereinafter, steady operation temperature) during detection of ammonia. In the ammonia detector 1 of the present embodiment, the temperature of the detection electrode 21 is preferably controlled to be in the range of 400° C. to 750° C. This inhibits ammonia from being consumed due to oxidation by the noble metal 211 in the detection electrode 21. Thus, ammonia is more accurately detected.

The current supply control section 58 controls the amount of current supply to the heating element 41 to control the temperature of the detecting element 2. The temperature of the detecting element 2 is detected using the temperature characteristics of the resistance (that is, the impedance) of the element components such as the solid electrolyte body 3 and the heating element 41. This eliminates the need for separately providing, for example, the temperature detecting element, which simplifies the structure of the device. The current supply control section 58 controls the heating by the heater 4 in accordance with an estimated value of the element temperature.

The ammonia detector 1 is constituted by mounting, for example, a non-illustrated cover or a housing on the detecting element 2. The ammonia detector 1 configured as described above is applied to, for example, an exhaust gas purifying system 100 illustrated in FIG. 5 and is located in an exhaust gas passage EX downstream of an SCR catalyst 101. A particulate filter F is located in the exhaust gas passage EX. An exhaust gas temperature sensor 102, an aqueous urea solution injector 103, and the SCR catalyst 101 are located downstream of the particulate filter F in this order. The particulate filter F collects particulate matter included in the exhaust gas emitted from an engine E. The SCR catalyst 101 constitutes a urea-SCR system, which causes NOx included in the exhaust gas to react with ammonia generated from the aqueous urea solution to reduce and purify NOx.

The ammonia detector 1 is mounted on the passage wall of the exhaust gas passage EX in a state in which, for example, the outer circumference of the detecting element 2 is held by a non-illustrated housing, and the distal end 18, which projects in the exhaust gas passage EX, is accommodated in an air-permeable cover. The ammonia detector 1 detects the concentration of ammonia in the exhaust gas G₁ that has passed through the SCR catalyst 101 without reacting with NOx. The detection result is output to an electronic control unit (ECU) 5 of the exhaust gas purifying system 100. The ECU 5 includes a sensor control section 50 (for example, refer to FIG. 1). The feedback on the detection result is reflected to the amount of aqueous urea solution to be supplied. Thus, the NOx purification reaction in the SCR catalyst 101 is efficiently performed.

The ammonia detector 1 is preferably of a mixed-potential type. In this case, the ammonia detector 1 outputs a mixed-potential signal in accordance with the concentration of ammonia. That is, the ammonia detector 1 includes a circuit 510, which outputs a mixed-potential signal. The detecting element 2 outputs a mixed-potential signal corresponding to, for example, a potential difference V between the detection electrode 21 and the reference electrode 22 based on the detection principle of the mixed-potential sensor. The detection electrode 21 simultaneously causes the following two electrochemical reactions at the interface between the detection electrode 21 and the solid electrolyte body 3. The electrochemical reactions include an electrochemical oxidation reaction involving ammonia to be detected (1) and an electrochemical reduction reaction involving oxygen (2). The reference electrode 22 causes the electrochemical reduction reaction involving oxygen (2).

2NH₃+3O₂−⇔N₂+3H₂O+6e⁻ (1)

O₂+4e⁻⇔2O²⁻ (2)

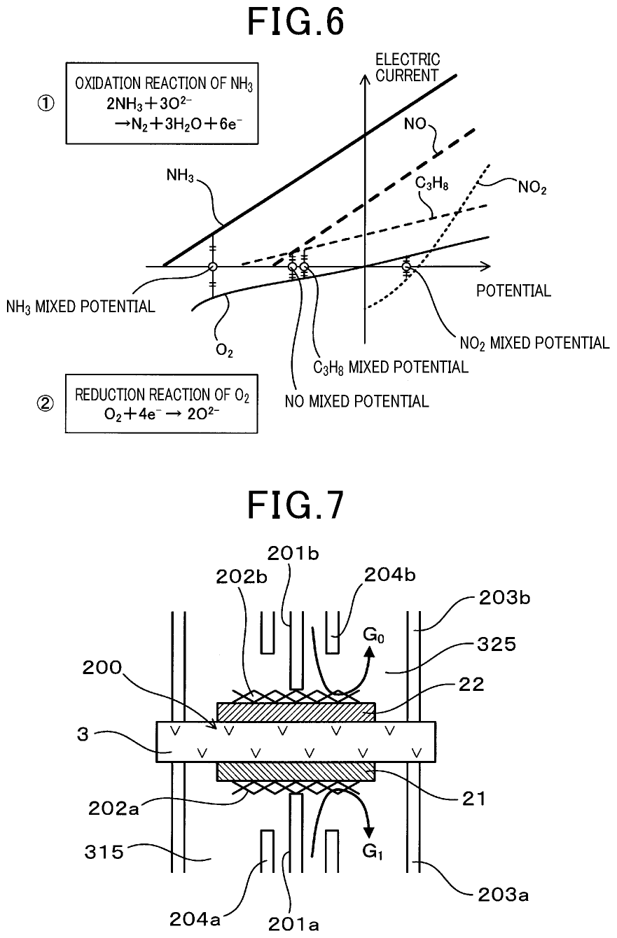

At this time, when the oxidation current caused by the electrochemical oxidation reaction (1) and the reduction current caused by the electrochemical reduction reaction (2) are balanced on the detection electrode 21, a mixed potential is established (see FIG. 6). That is, the potential of the detection electrode 21 is determined in accordance with the mixed potential of these two electrochemical reactions, and the potential difference V with respect to the reference electrode 22 is retrieved as the sensor output. The sensor output is input to a concentration calculator 52 of the sensor control section 50 as required, and the concentration calculator 52 calculates the concentration of ammonia. The sensor output may be the potential difference or a value of current that flows based on the potential difference. That is, a current may be allowed to flow between the electrodes to detect a voltage, or a voltage may be applied between the electrodes to detect a current.

Furthermore, the ammonia detector 1 for use in vehicles is used to detect, for example, the concentration of ammonia in the exhaust gas G₁ generated by the urea-SCR system. In this case, the concentration of a minute amount of ammonia in the exhaust gas G₁ needs to be accurately detected while the concentration of each gas component in the exhaust gas G₁ is easily changed. However, for example, in the ammonia detector 1 of a limited-current type, the detection electrode 21 oxidizes ammonia during application of an electric field, which is likely to cause a baseline to fluctuate. As a result, the concentration of a minute amount of ammonia that should be detected fluctuates depending on the baseline. For this reason, the ammonia detector 1 of a mixed-potential type has been studied.

Unfortunately, the detection electrode 21 with the conventional configuration formed of Au or an alloy of Pt and Au has a high ammonia oxidation activity. As a result, even in the mixed-potential ammonia detector 1, the electrode material itself of the detection electrode 21 may possibly oxidize ammonia and fluctuate the baseline.

Since the main component of the noble metal 211 is Pd in the detection electrode 21 of the present embodiment, the ammonia oxidation activity is low. Thus, the electrode material itself of the detection electrode 21 is inhibited from oxidizing ammonia, which inhibits the fluctuation of the baseline even in the mixed-potential ammonia detector 1. Consequently, the concentration of ammonia is accurately detected even in the mixed-potential ammonia detector 1.

The ammonia detector 1 preferably includes a potential difference detector 51. In this case, the potential difference detector 51 detects the mixed potential. The potential difference detector 51 detects the potential difference between the detection electrode 21 and the reference electrode 22. Additionally, the ammonia detector 1 includes the concentration calculator 52. The concentration calculator 52 calculates the concentration of ammonia in the measured gas G₁ based on the potential difference detected by the potential difference detector 51.

In the ammonia detector 1 of the present embodiment, the detection electrode 21 includes 50% or more by mass of Pd as the noble metal 211. The noble metal 211 configured like this has a high melting point and is capable of maintaining the electrode performance under high-temperature environments such as 1400° C.

Thus, the ammonia detector 1 can be formed by integrally firing the detection electrode 21 and other members during manufacturing. Other members include, for example, the reference electrode 22, the solid electrolyte body 3, the heater 4, and the insulating substrate 29. This allows the ammonia detector 1 to be manufactured at a low cost. Additionally, evaporation or the like of the electrode components from the detection electrode 21 is inhibited, and the heat resistance of the detection electrode 21 is high. That is, the ammonia detector 1 is excellent in durability under high-temperature environments.

Furthermore, the detection electrode 21 inhibits consumption of ammonia due to, for example, excessive oxidation while maintaining the reactivity to ammonia to the degree that the concentration of ammonia in the exhaust gas G₁ can be accurately detected. As a result, since the ammonia detector 1 detects ammonia while inhibiting consumption of ammonia on the detection electrode 21, the concentration of ammonia is accurately detected.

The reason why the concentration of ammonia can be accurately detected may also be as follows. That is, since the detection electrode 21 contains alumina that does not have oxygen ion conductivity, NO₂ is likely to be decomposed to NO on the surface or the inside of the detection electrode 21. NO₂ is thus hindered from reaching the three-phase interface 314A between the detection electrode 21 and the solid electrolyte body 3, and NO generated by the decomposition of NO₂ reaches the three-phase interface 314A. Since the detection electrode 21 contains 50% or more by mass of Pd, the sensitivity to NO is decreased while the sensitivity to ammonia is increased. Thus, the ammonia detector 1 accurately detects the concentration of ammonia even under an environment where, for example, NOx coexists.

### Experimental Example 1

The present example is a comparative evaluation of the performance of the noble metal electrode. First, noble metal electrodes formed of Pd, Rh, Au, Ir, and Pt were used to prepare simplified elements 20, and the performance of the simplified elements 20 in detecting ammonia was compared and evaluated. The reference signs used in and after the present experimental example that are the same as the reference signs of the above embodiments represent, unless otherwise specified in particular, the same components as those in the above embodiments.

As illustrated in FIG. 7, each simplified element 20 includes the solid electrolyte body 3, the detection electrode 21, and the reference electrode 22. The solid electrolyte body 3 is disk-shaped. The detection electrode 21 and the reference electrode 22 are located on the surfaces of the solid electrolyte body 3 opposite to each other with the solid electrolyte body 3 located in between.

The solid electrolyte body 3 is formed of YSZ. The detection electrode 21 is formed of the noble metal including Pd, Rh, Au, Ir, or Pt and YSZ. The reference electrode 22 is formed of Pt and YSZ.

A current collector 202a to which a lead wire 201a is welded is mounted on the detection electrode 21, and a current collector 202b to which a lead wire 201b is welded is mounted on the reference electrode 22. The lead wires 201a and 201b are formed of Pt wires, and the current collectors 202a and 202b are formed of Pt meshes.

In manufacturing each simplified element 20, first, the electrode material for the detection electrode 21 was screen printed on one of the sides of the solid electrolyte body 3, and the electrode material for the reference electrode 22 was screen printed on the other side of the solid electrolyte body 3. Subsequently, the electrode materials were fired to form the detection electrode 21 and the reference electrode 22 on the solid electrolyte body 3. Next, the current collector 202a to which the lead wire 201a is welded was mounted on the detection electrode 21, and the current collector 202b to which the lead wire 201b is welded was mounted on the reference electrode 22.

The evaluation device illustrated in FIG. 7 was used for the evaluation. In preparing the evaluation device, first, a cap 203a was mounted on the surface of the solid electrolyte body 3 (of the simplified element 20) on which the detection electrode 21 is formed to define a measured gas chamber 315. A cap 203b was mounted on the surface of the solid electrolyte body 3 (of the simplified element 20) on which the reference electrode 22 is formed to define the reference gas chamber 325. A gas pipe 204a through which the measured gas G₁ flows was inserted in the cap 203a, which covers the detection electrode 21. A gas pipe 204b through which the reference gas G₀ flows was inserted in the cap 203b, which covers the reference electrode 22. The lead wires 201a and 201b were connected to the potential difference detector 51, which is located outside, and the potential difference detector 51 was used to read the potential difference between the detection electrode 21 and the reference electrode 22.

In the evaluation, the exhaust gas G₁ was fed through the gas pipe 204a, and the atmospheric air G₀ was fed through the gas pipe 204b to measure the potential between the detection electrode 21 and the reference electrode 22. The gas temperature was 450° C. In addition to containing 5% by volume of O₂ with respect to the nitrogen gas, which is the basic component, there were a case in which the exhaust gas G₁ contains no gas to be measured, a case in which the exhaust gas G₁ contains 100 ppm by volume of gas to be measured, and a case in which the exhaust gas G₁ contains 200 ppm by volume of gas to be measured. The gas flow rate was 300 ml/min. The gas to be measured was NH₃, NO, or NO₂.

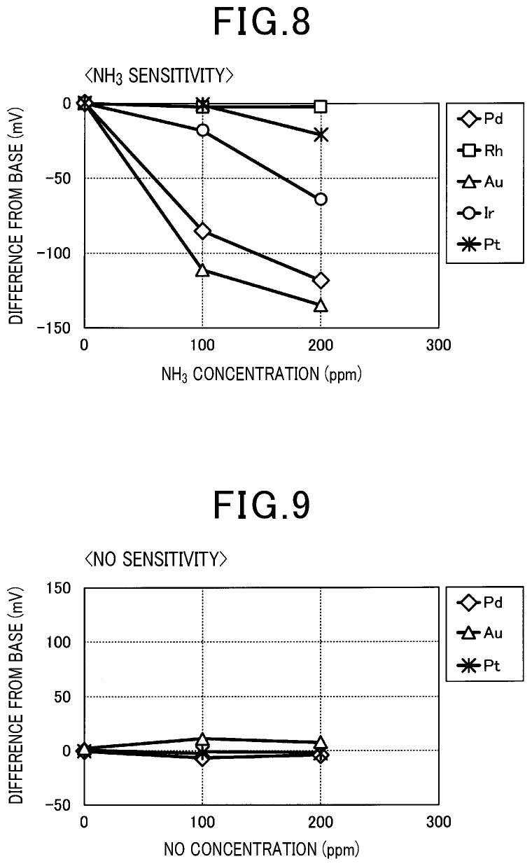

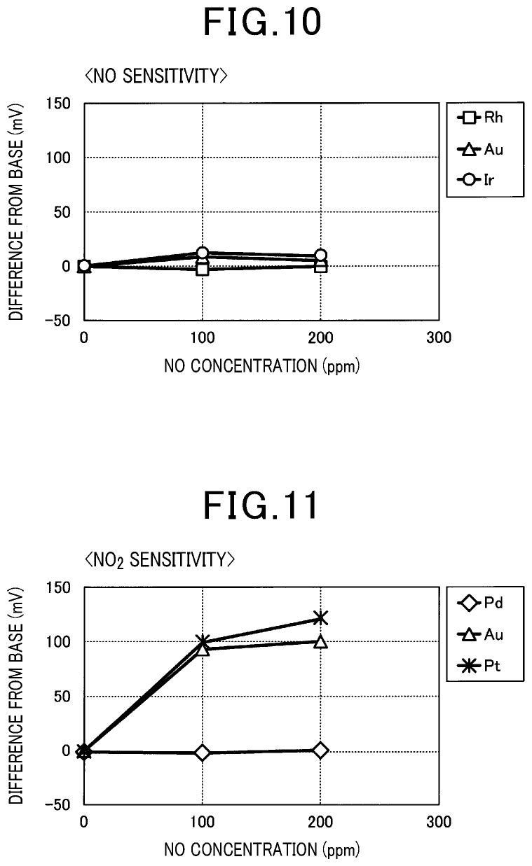

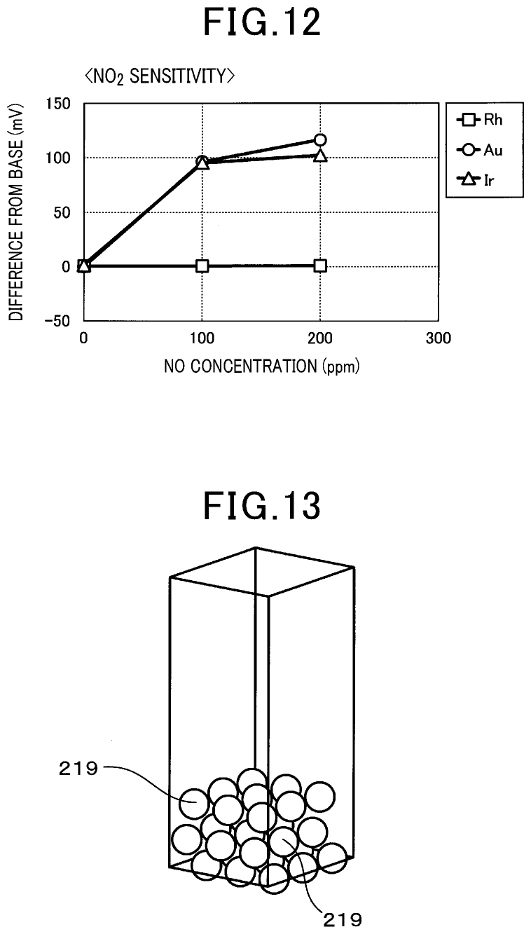

The sensitivity to the measured gas G₁ when each noble metal is used for the detection electrode 21 is shown in FIGS. 8 to 12. The vertical axis in FIGS. 8 to 12 represents the difference between the potential of the detection electrode 21 and the base potential, which is the reference electrode 22. The greater the difference is, the higher becomes the sensitivity. FIGS. 9 and 10 showing the NO sensitivity and FIGS. 11 and 12 showing the NO₂ sensitivity show the result of Au alongside for comparison.

As apparent from FIG. 8, Pd has a sufficiently higher sensitivity to ammonia than other noble metals. In the meantime, as apparent from FIGS. 9 to 12, Pd has a lower sensitivity to NO and NO₂ than Au. In particular, the sensitivity of Pd to NO₂ is sufficiently lower than that of Au and other noble metals.

In contrast, Pt, Rh, and Ir have a significantly lower sensitivity to ammonia than Au. In particular, the decrease in the sensitivity to a low concentration ammonia is more significant. Pt has a particularly higher sensitivity to NO₂ than other noble metals. Although Rh has a low sensitivity to NO and NO₂, the sensitivity to ammonia is also very low. Ir has a high sensitivity to NO and NO₂.

As described above, while Pd has a sufficiently high sensitivity to ammonia, the sensitivity to NO and NO₂ is sufficiently lower than other noble metals. In other words, Pd has an excellent selectivity to ammonia and is suitable for the detection electrode 21 of the ammonia detector 1.

Subsequently, to study the noble metal in terms of the consumption of ammonia due to oxidation, the O₂ dissociative adsorption energy of the noble metal was simulated for evaluation. The O₂ dissociative adsorption energy is a value indicating, for example, how easily O₂ is adsorbed and the degree of adsorbability. The degree of adsorbability to each noble metal may also be described as how difficult it is to separate from each noble metal. Since O₂ is adsorbed by the noble metal in a state in which O₂ is dissociated into O atoms, the dissociative adsorption energy of O₂ was used as an index.

Calculation of the O₂ dissociative adsorption energy by simulation was performed using analysis software D mol³ manufactured by DASSAULT SYSTEMES BIOVIA K.K. based on first-principles calculation. A Generalized Gradient Approximation-Perdew Burke Ernzerhof (GGA-PBE) function was used. The specific calculation method is as follows.

First, base atoms 219 were selected to configure a simulation model illustrated in FIG. 13. The base atoms 219 are any of Au, Pt, Rh, and Pd. These base atoms 219 all form a face-centered cubic lattice in a stable manner. The expression “base atoms” is not necessarily correct since the calculation of the O₂ dissociative adsorption energy is performed on a simulation model of a single noble metal and not an alloy of noble metals. However, the expression “base atoms” is used herein to describe the simulation model substituted by substitution elements below.

In calculating the O₂ dissociative adsorption energy, the conditions of the simulation model illustrated in FIG. 13 are as follows.

Number of cells: 3×3

Number of layers: 3 (second layer and third layer are fixed)

Vacuum layer: 20 Å

Surface: fcc (111) surface

The simulation model includes adsorption sites as illustrated in FIG. 14. In FIG. 14, an adsorption site T is referred to as “on-top” and represents a onefold coordinated on-top site. An adsorption site B is referred to as “bridge” and represents a twofold coordinated bridge site. Adsorption sites H are referred to as “hollow” and represent threefold coordinated hollow sites. The adsorption sites H further include two kinds including a first adsorption site H1 and a second adsorption site H2. The first adsorption site H1 is referred to as “hcp hollow” and represents a hexagonal close-packed hollow site below which an atom forming a second layer exists. The second adsorption site H2 is referred to as “fcc hollow” and represents a face-centered cubic hollow site below which there is no atom forming a second layer.

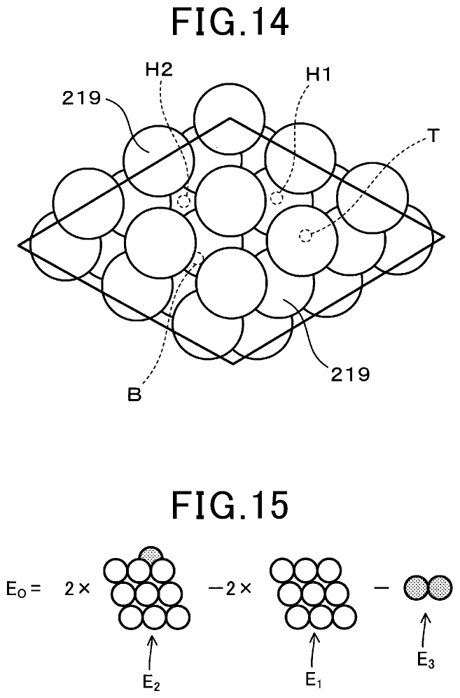

The O₂ dissociative adsorption energy obtained by simulation is free energy when an oxygen atom O is located in the adsorption site T, B, H1, or H2 as illustrated in FIG. 14. The energies at the adsorption sites T, B, H1, and H2 are calculated, and the greatest energy is used in the following expression. The expression of the O₂ dissociative adsorption energy Eo is represented by the following equation (I), and the calculation model is illustrated in FIG. 15. The white circles in FIG. 15 represent the base atoms, circles with dots represent oxygen atoms.

E₀=2×E₂−2×E₁−E₃ (I)

In equation (I), E₁ to E₃ represent the following energies.

E₁: energy with only base atoms

E₂: energy when an oxygen atom O is placed on base atoms

E₃: energy with only O₂

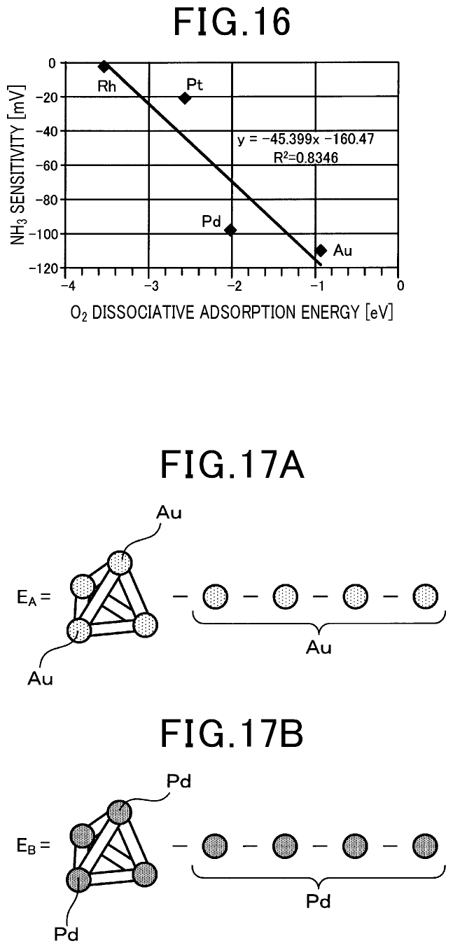

The O₂ dissociative adsorption energy of each noble metal is shown in FIG. 16. FIG. 16 shows the relationship between the O₂ dissociative adsorption energy of each noble metal obtained by simulation and the NH₃ sensitivity of each noble metal. The NH₃ sensitivity of each noble metal is the result obtained when the concentration of ammonia is 100 ppm as described above with reference to FIG. 8.

FIG. 16 indicates that the oxidizing power becomes stronger as the absolute value of the O₂ dissociative adsorption energy is increased, that is, leftward in the direction of the lateral axis from 0. In the meantime, the sensitivity increases as the absolute value of the NH₃ sensitivity increases, that is, downward in the direction of the vertical axis from 0.

As apparent from FIG. 16, there is a correlation between the O₂ dissociative adsorption energy and the NH₃ sensitivity, and the ammonia sensitivity is likely to increase as the O₂ dissociative adsorption energy decreases. As apparent from FIG. 16, Pd and Au have high ammonia sensitivity and weak oxidizing power. In contrast, Pt and Rh have low ammonia sensitivity and strong oxidizing power. For this reason, the detection electrode 21 formed of Pt and Rh has a stronger oxidizing power and causes ammonia to be easily oxidized and consumed before reacting at the three-phase interface 314A. That is, in this case, it is difficult to accurately detect ammonia since reaction that does not contribute to the potential occurs. Since the ammonia detector 1 for use in vehicles that is required to detect the concentration of a minute amount of ammonia particularly requires high detection accuracy, Pd, Au, or an alloy of Pd and Au is more effective for the detection electrode 21.

Next, Au and Pd are studied in terms of cohesive energy. The cohesive energy is the energy necessary for separating atoms or ions in a cohesive state from each other. That is, the cohesive energy is an index representing how easily evaporation occurs or how difficult it is to evaporate. In the present example, the cohesive energy of Au and Pd is calculated.

Like the O₂ dissociative adsorption energy obtained by simulation described above, the cohesive energy was calculated by creating a simulation model and performing the first-principles calculation using the analysis software. The setting conditions of the analysis software were as follows.

Functiona: GGA-PBE

Spin polarization: unrestricted

Core treatment: all-electron relativistic

FIG. 17A shows a calculation model of a cohesive energy E in a simulation model of only Au atoms. That is, a cohesive energy EA of Au is the energy obtained by subtracting the energy of Au atoms constituting the Au base material from the energy of the Au base material. FIG. 17B shows a calculation model of the cohesive energy E in a simulation model of only Pd atoms. That is, a cohesive energy E_{B} of Pd is the energy obtained by subtracting the energy of Pd atoms constituting the Pd base material from the energy of the Pd base material. The results of the cohesive energies are shown in FIG. 18.

As apparent from FIG. 18, the cohesive energy of Pd is higher than the cohesive energy of Au. This indicates that Pd is less likely to evaporate than Au. Thus, Pd is suitable for the detection electrode 21 of the ammonia detector 1 in terms that the electrode performance is inhibited from being decreased even under high-temperature environments and in terms that Pd can be fired integrally at a high temperature.

### Experimental Example 2

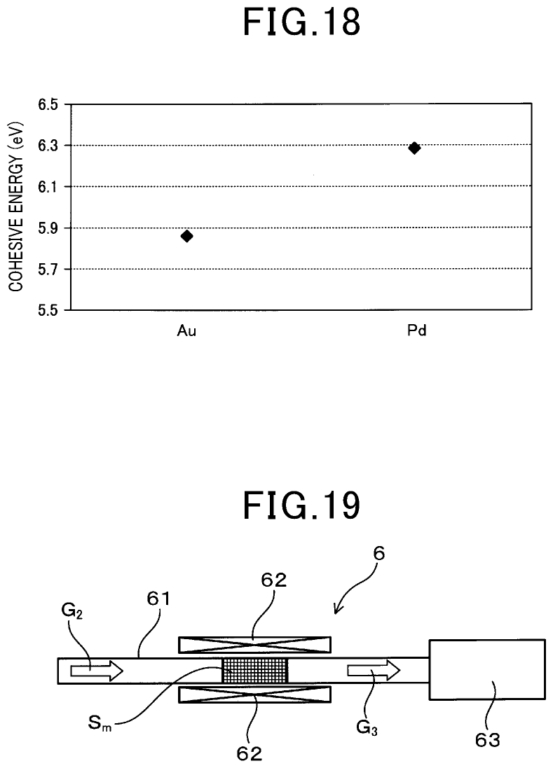

The present example compares the oxidizing power of Pd and Au with respect to ammonia. In the present example, as illustrated in FIG. 19, the oxidizing power of Pd and the oxidizing power of Au were actually compared and evaluated by a temperature-programmed reaction method (TPR).

As illustrated in FIG. 19, an evaluation device 6 used in this example includes a sample pipe 61, a heater 62, which heats the inside of the sample pipe 61, and a mass spectrometer 63. The mass spectrometer 63 is a Fourier transform infrared spectrophotometer. The sample pipe 61 is filled with noble metal powder S_{M} that is to be measured. The noble metal powder S_{M} is Pd powder or Au powder.

In the evaluation, an introduction gas G₂ was fed into the sample pipe 61 in one direction, and the noble metal powder S_{M} was supplied through the sample pipe 61. The introduction gas G₂ contained 2000 ppm by volume of NH₃ and 5000 ppm by volume of O₂ with respect to He. The flow rate of the introduction gas G₂ was 300 ml/min. The passing of the introduction gas G₂ caused the following oxidation reaction of ammonia (3) on the noble metal powder S_{M} in the sample pipe 61.

4NH₃+3O₂→2N₂+6H₂O (3)

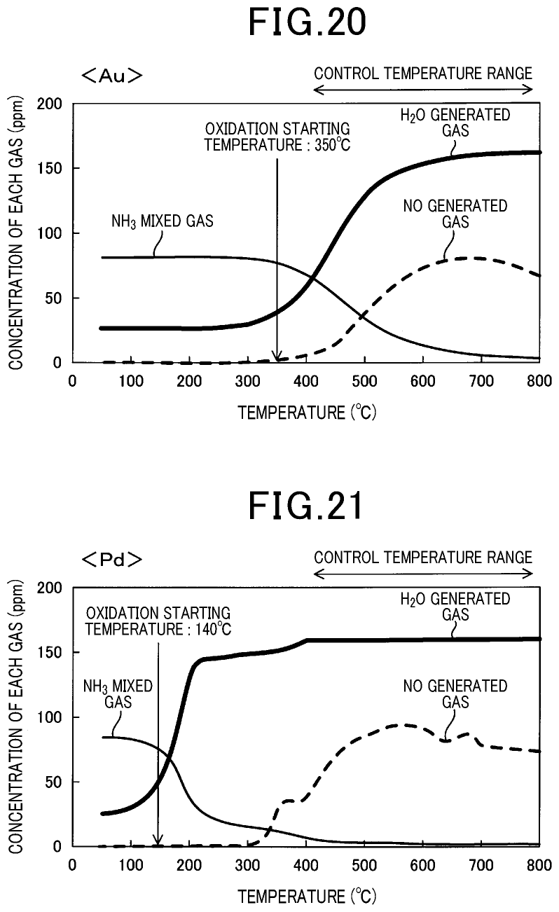

Next, the reacted gas G₃ that has passed through the noble metal powder S_{M} was analyzed by the mass spectrometer 63 located at a downstream section. FIGS. 20 and 21 show the results of the gas concentration for Au and Pd detected by the mass spectrometer 63 when the inside of the sample pipe 61 was heated at a temperature increase speed of 20° C./min. In FIGS. 20 and 21, the temperature at which the oxidation starts is the temperature at which NH₃ starts to decrease.

As apparent from FIGS. 20 and 21, like the result of the simulation in Experimental Example 1, Pd starts oxidation at a lower temperature and has a stronger oxidizing power to ammonia compared with Au. Additionally, Pd has good selectivity and sensitivity to ammonia. In contrast, Au has a weak oxidizing power to ammonia. This indicates that an alloy of Pd and Au will achieve good selectivity and sensitivity to ammonia while reducing the oxidizing power to ammonia.

To improve the detection accuracy of the ammonia detector 1, the oxidation of ammonia before reaching the three-phase interface 314A of the detection electrode 21 is preferably inhibited. For this purpose, for example, the following conditions (A) to (C) are effective.

(A) Reduce the thickness of the detection electrode 21 to the range of, for example, 1 to 20 μm, and preferably to the range of 1 to 10 μm. This will be described in more detail below in Experimental Example 5.

(B) Set the control temperature of the detection electrode 21 to 400 to 750° C. as described in the first embodiment. The control temperature lower than 400° C. causes NO₂ to reach the three-phase interface 314A without being decomposed, deteriorating the selectivity of the detection electrode 21 to NO₂. In contrast, the control temperature exceeding 750° C. causes ammonia to oxidize before reaching the three-phase interface 314A, decreasing the amount of oxidation at the three-phase interface 314A. This decreases the sensitivity of the detecting element 2 to ammonia.

(C) Adjust the average particle size of the noble metal 211 to the range of, for example, 0.2 to 5 μm as described in the first embodiment.

### Experimental Example 3

The present example examines the sensitivity of the detection electrode 21 to ammonia, NO, and NO₂ when Pd or Au is used for the noble metal 211. The sensitivity to each gas was measured in the same manner as in Experimental Example 1. Note that, in the present example, the oxygen concentration of a test gas that contains each gas (exhaust gas G₁) was 10% by volume.

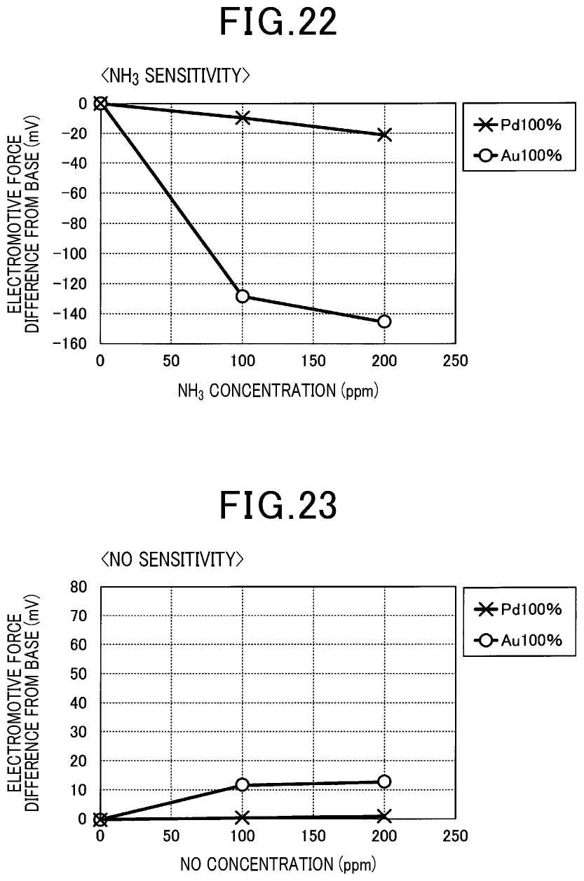

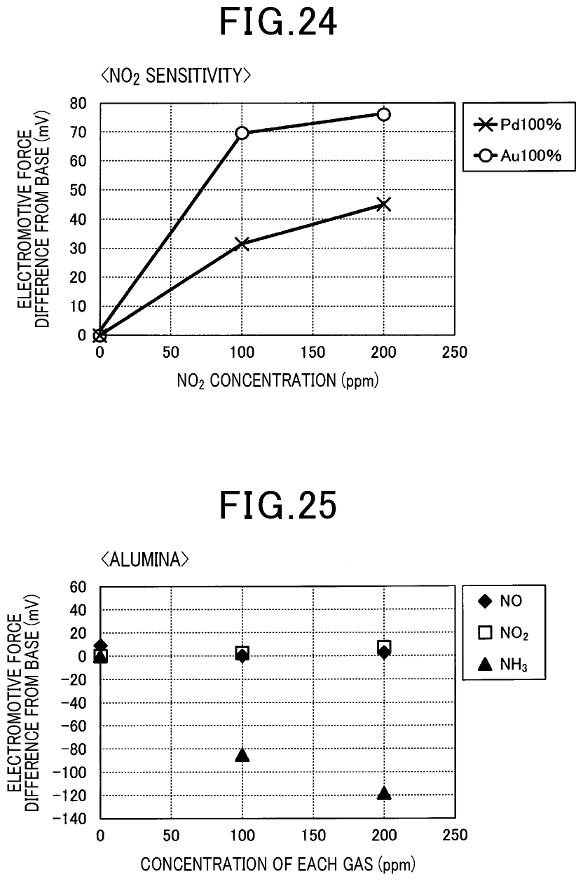

The sensitivity of the detection electrode 21 made of 100% by mass of Pd and the sensitivity of the detection electrode 21 made of 100% by mass of Au with respect to each gas were measured. The results are shown in FIGS. 22 to 24. In FIGS. 22 to 24, “%” indicates “% by mass”. In each figure, the electromotive force difference from the base represents the potential difference between the detection electrode 21 and the reference electrode 22.

As apparent from FIGS. 22 to 24, although Au has an excellent sensitivity to ammonia, the sensitivity to NO and NO₂ is also high. This means that the selectivity to ammonia is insufficient, and for example, the mixed-potential ammonia detector 1 undesirably reflects the concentration of NO and NO₂ in the mixed potential in addition to the concentration of ammonia.

In the meantime, although Pd has a lower sensitivity to ammonia than Au, it has little sensitivity to NO. Au and Pd both have a high sensitivity to NO₂. In other words, with Pd being used for the noble metal 211 of the detection electrode 21, NO is not detected by the ammonia detector 1 even if NO₂ is decomposed to NO on the detection electrode 21, and NO then reaches the three-phase interface 314A between the solid electrolyte of the solid electrolyte body 3, the noble metal 211, and the gas phase. This means that the existence of NOx such as NO₂ does not deteriorate the detection accuracy of ammonia.

As described above, the present example indicates that Pd is excellent in the selectivity to ammonia and is effective as the noble metal 211 of the detection electrode 21 in the ammonia detector 1.

### Experimental Example 4

The present example examines the influence of an additive component other than the noble metal 211 in the detection electrode 21. First, Pd, an additive component, and a vehicle (such as a solvent) were combined and were mixed by a mixer to prepare electrode pastes shown in Table 1. Alumina or YSZ was used for the additive component.

TABLE 1

| Kinds of additive | | |
|---|---|---|
| component | Alumina | YSZ |
| Content rate of Pd | 76.68 | 72.53 |
| [% by mass] | | |
| Ratio of additive | 9.2 | 14.51 |
| component [% by mass] | | |
| Content of vehicle | 14.11 | 12.96 |
| [% by mass] | | |
| Viscosity [Pa · s] | 210 | 150 |
| Average particle | 2.2 | 1.2 |
| diameter of Pd [μm] | | |
| Specific surface area | 0.84 | 1.1 |
| [m²/g] | | |

Next, each electrode paste shown in Table 1 was used as the electrode material for the detection electrode 21, and the simplified element 20 that is the same as the one in Experimental Example 1 was prepared. The simplified element 20 is the same as the one in Experimental Example 1 except that the electrode material of the detection electrode 21 was changed.

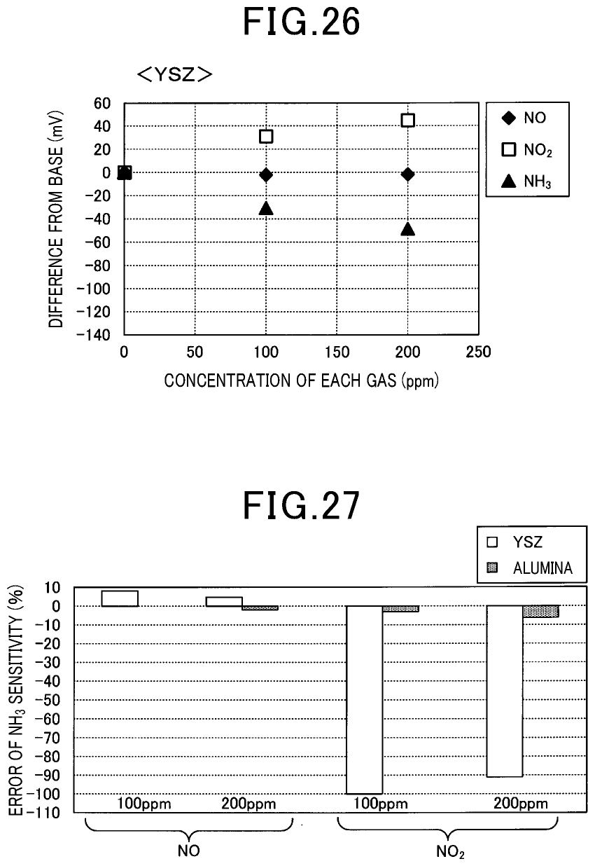

Subsequently, in the same manner as Experimental Example 1, the evaluation device was prepared, and the exhaust gas G₁ and the atmospheric air G₀ were fed through the gas pipe. The potential difference between the detection electrode 21 and the reference electrode 22 at this time was measured. The gas temperature was 450° C., and the gas flow rate was 300 ml/min. The present example includes, in addition to containing 10% by volume of O₂ with respect to the nitrogen gas, which is the basic component, a case in which the exhaust gas G₁ contains no gas to be measured, a case in which the exhaust gas G₁ contains 100 ppm by volume of the gas to be measured, and a case in which the exhaust gas G₁ contains 200 ppm by volume of the gas to be measured. The gas to be measured was NH₃, NO, or NO₂. The results are shown in FIGS. 25 and 26.

Based on the results shown in FIGS. 25 and 26, the percentage of the sensitivity of the detection electrode 21 to NO to the sensitivity of the detection electrode 21 to ammonia was calculated as the error (%) of the ammonia sensitivity caused by NO. Furthermore, the percentage of the sensitivity of the detection electrode 21 to NO₂ to the sensitivity of the detection electrode 21 to ammonia was calculated as the error (%) of the ammonia sensitivity caused by NO₂. The results are shown in FIG. 27.

The error (%) of the ammonia sensitivity caused by NO and NO₂ was obtained as follows.

Error (%)=(electromotive force difference with respect to NO or NO₂−electromotive force difference with respect to ammonia)/electromotive force difference with respect to ammonia×100(%)

As apparent from FIG. 26, the detection electrode 21 that uses YSZ as the additive component shows the potential difference with respect to NO₂ besides ammonia. Consequently, as apparent from FIG. 27, the error of the ammonia sensitivity is great.

In contrast, as apparent from FIG. 25, the detection electrode 21 that uses alumina as the additive component shows little potential difference with respect to NO and NO₂, while showing sufficient potential difference with respect to ammonia. Consequently, as apparent from FIG. 27, the error of the ammonia sensitivity is small.

As described above, according to the present example, the detection electrode 21 that contains alumina as the additive component has a decreased sensitivity to NO and NO₂, which further improves the detection accuracy to ammonia. In the present example, as described above, the influence of the additive component on the detection electrode 21, which contains Pd as the noble metal 211, was examined. The same result will be obtained with the detection electrode 21 that contains an alloy of Pd and Au with Pd included as the main component.

Although embodiments of the present disclosure have been described as above, the present disclosure is not limited to the illustrated embodiments, but may be applied to various embodiments without departing from the scope of the disclosure. For example, in the first embodiment, although the ammonia detector 1, which includes the detection electrode 21 for detecting ammonia, was described, the ammonia detector 1 may further include a detection electrode for detecting NOx such as NO and NO₂. In this case, the ammonia detector 1 detects the concentration of NOx in addition to the concentration of ammonia.

In other words, the ammonia detector 1 may include the ammonia detection section, which detects at least ammonia, and additionally a gas detection section, which detects gas other than ammonia. The ammonia detection section is constituted by, for example, a first detection electrode for detecting ammonia, a reference electrode, and a solid electrolyte body on which these electrodes are formed. In the meantime, the gas detection section is constituted by, for example, a second detection electrode for detecting gas other than ammonia, a reference electrode, and a solid electrolyte body on which these electrodes are formed.

The ammonia detection section and the gas detection section may share a single reference electrode or may respectively include different reference electrodes such as a first reference electrode and a second reference electrode. The above is also applicable to the solid electrolyte body. The ammonia detection section and the gas detection section may share a single solid electrolyte body or may respectively include different solid electrolyte bodies such as a first solid electrolyte body and a second solid electrolyte body. Furthermore, the ammonia detector 1 can include two or more detection electrodes for detecting gases other than ammonia.

Although the present disclosure has been described in accordance with the embodiments, it is understood that the present disclosure is not limited to the embodiments and the configurations. The present disclosure embraces various modifications and deformations that come within the range of equivalency. Additionally, various combinations and forms, or other combinations and forms including only one or more additional elements, or less than all elements are included in the scope and ideas obtainable from the present disclosure.

What is claimed is:

1. An ammonia detector comprising:
a solid electrolyte body including a measured gas facing surface, which comes into contact with a measured gas, and a reference gas facing surface, which comes into contact with a reference gas;
a detection electrode formed on the measured gas facing surface of the solid electrolyte body; and
a reference electrode formed on the reference gas facing surface of the solid electrolyte body, wherein
the detection electrode contains at least alumina and 50% or more by mass of Pd.

2. The ammonia detector according to claim 1, wherein the ammonia detector is of a mixed-potential type.

3. The ammonia detector according to claim 1, wherein the detection electrode has a thickness of 1 μm to 20 μm.

4. The ammonia detector according to claim 1, wherein the measured gas facing surface is a first main surface of the solid electrolyte body, which is plate-shaped, and the reference gas facing surface is a second main surface of the solid electrolyte body opposite to the first main surface.

5. The ammonia detector according to claim 1, wherein the detection electrode contains a solid electrolyte component which is identical to material constituting the solid electrolyte body, and
the content of alumina in the detection electrode is greater than the content of the solid electrolyte component in the detection electrode.

6. The ammonia detector according to claim 1, wherein the detection electrode substantially contains no solid electrolyte component which configures the solid electrolyte body.

7. The ammonia detector according to claim 1, further comprising a potential difference detector, which detects the potential difference between the detection electrode and the reference electrode, and a concentration calculator, which calculates the concentration of ammonia in the measured gas based on the potential difference detected by the potential difference detector.

8. The ammonia detector according to claim 1, further comprising a heater including a heating element, which generates heat upon energization, and a current supply control section, which controls the amount of current supply to the heating element for the temperature of the detection electrode to be in the range of 400° C. to 750° C.

9. The ammonia detector according to claim 1, wherein the average particle size of Pd in the detection electrode is 0.2 μm to 5 μm.
